(19) United States
(12) Patent Application Publication
(10) Pub. No.: US 2024/0000940 A1
(43) Pub. Date: Jan. 4, 2024

(54) POROUS, HIGH-Z AND CARBON-FREE PARTICLES AS RADIOENHANCERS

(71) Applicant: NANOBIOTIX S.A., PARIS (FR)

(72) Inventors: LAURENT LEVY, PARIS (FR); CÉLINE BERJAUD, PARIS (FR); ALEXANDRA MADEIRA, PARIS (FR); JULIE MARILL, PARIS (FR); MARIE-EDITH MEYRE, PARIS (FR)

(21) Appl. No.: 18/039,728

(22) PCT Filed: Nov. 30, 2021
(86) PCT No.: PCT/EP2021/083469

§ 371 Date: Jun. 1, 2023

(30) Foreign Application Priority Data
Dec. 1, 2020 (EP) 20306470.4

(51) Int. Cl. A61K 41/00; A61P 35/00; A61N 5/10; A61K 33/42; B82Y 5/00

(52) U.S. Cl. CPC A61K 41/0038; A61P 35/00; A61N 5/1001; A61K 33/42; B82Y 5/00; A61N 2005/1098

(57) ABSTRACT

The invention concerns a particle and a composition comprising particles and their use in oncology. Specifically, the particles are porous, high-Z and carbon-free particles having internal pores, of longest dimension of at least 0.5 nm, and are for use in altering or destroying target cells in a mammal when said cells are exposed to ionizing radiation.

The present invention also provides a porous, high-Z and carbon-free particle wherein at least part of the porous structure of the particle is occupied by at least one therapeutic agent which is preferably for use in oncology.

### BACKGROUND

Radioenhancers, when exposed to ionizing radiation, amplify the radiation dose deposit locally (on-off activity). When present at the cancerous tumor cell level and exposed to ionizing radiation, they augment tumor cell damage and destruction, compared to tumor cells without radioenhancers, without additional toxicity to the surrounding healthy tissues.

One of the key criteria to ensure an efficient and safe use of these radioenhancers is that they should be stable (i.e., they should not undergo any degradation, typically via bond cleavage) both in vivo and under exposure to ionizing radiation, particularly in the context of repeated radiotherapy (RT) sessions.

Metal Organic Frameworks (MOFs) are well-established porous compounds, well-known for their use in various fields such as gas storage and separation, liquid separation and purification, catalysis and sensing. Recently, these Metal Organic Frameworks (MOFs), together with Metal Organic Plates (MOPs) or Metal Organic Layers (MOLs) bearing high-Z elements have been proposed as porous radioenhancers [WO2016061256, WO2019028250]. These MOFs, MOPs or MOLs are generally constructed by assembling metal oxo-clusters of high-Z elements using organic linkers. The high-Z element is responsible for efficient interaction with ionizing radiation, leading to enhanced radiation dose deposit. Assembly of these metal oxo-clusters with organic linkers creates pores in the framework, into which small molecule drugs, or other compounds can be trapped for use in vivo.

Furthermore, these compounds offer the possibility to easily tune their composition by changing the metal and/or the organic linker.

Also, the organic linkers themselves may possess specific characteristics that allow additional therapeutic or other useful mechanisms of actions—such as the photodynamic therapy—that can act synergistically with the radioenhancing effect provided by the high-Z metal oxo-clusters.

To date, many MOFs, MOPs or MOLs use carboxylate groups as the coordinating groups between the metal-oxo clusters and the organic linkers because, presumably, carboxylate groups have well-studied coordination geometries giving predictable framework topologies. However, the latter systems present a high risk of framework degradation in vivo as in vivo (hydrolytic) cleavage between organic linkers and the metal oxo-clusters has been reported [Kathryn E. deKrafft et al. Zr- and Hf-based nanoscale metal-organic frameworks as contrast agents for computed tomography. J Mater Chem. 2012 January 1; 22 (35)]. In view of the intended use of these systems as radioenhancers in vivo, framework degradation will trigger a, most likely, unfavorable change of compound chemico-physical properties, as well as the possible generation of secondary undefined species, thus bearing a toxicological risk.

Moving away from these carboxylate-based organic linkers, phosphonate-based organic linkers have been proposed to increase the (hydrolytic) stability between the high-Z metal oxo-clusters and the organic linkers, despites of the often-poor crystallinity of the resulting compound [B. Gelfand, J. Lin, G. Shimizu. Development of Phosphonate Monoesters Building Units in Metal-Organic Frameworks. Acta Cryst. (2014), A70, C1127]. However, the phosphorus-carbon (P—C) bond from phosphonate compounds has been shown to undergo photodegradation and P—C bond cleavage triggered by Reactive Oxygen Species (ROS) [Congcong Xia et al. Mechanism of methylphosphonic acid photodegradation based on phosphate oxygen isotopes and density functional theory. RSC Adv., 2019, 9, 31325-31332].

Therefore, the stability of MOFs, MOPs or MOLs, in particular, under ionizing radiation conditions, and especially where the patient undergoes multiple RT sessions, is questioned.

Indeed, typical radiation therapy (also known as radiotherapy, or RT) approaches require several treatment sessions per week (usually programmed Monday to Friday depending on hospital/clinic working days) and this schedule usually continues for, typically, 3 to 9 weeks. Radiotherapy protocols are established according to the patient cancer type and clinical staging [AJCC CANCER STAGING MANUAL Seventh Edition], the patient's health status, etc. [see, typically, the “Principles of Radiotherapy” in the NCCN GUIDELINES for the treatment of cancer by site such as for example Head & Neck Cancers, Esophageal and Esophagogastric Junction Cancers, Central Nervous System Cancers, Non-Small Cell Lung Cancer, Pancreatic Adenocarcinoma, Prostate Cancers, Rectal Cancers, etc.].

Typical radiation therapy protocols, for example, in head and neck cancers, rectal cancers, esophageal cancers, non-small cell lung cancers, or central nervous system cancers are established with external beam radiotherapy (typically IMRT and high energy (≥4 MV) photons beam) with the following regimes, depending whether it is used for a definitive treatment, a palliative treatment, a concurrent or sequential systemic therapy/radiotherapy, a pre-operative radiotherapy, a post-operative radiotherapy:
Conventional Fractionation: from, typically, 1.6 Gy up to 2.25 Gy per fraction (for example, 1.6, 1.8, 2.0, 2.12, 2.25 Gy per fraction), daily (Monday to Friday), over 5 weeks up to 7 weeks;
Hyperfractionation: with, typically, 1.2 Gy per fraction, twice daily, over 7 weeks (for example, from 79.2 Gy up to 81.6 Gy over 7 weeks, twice daily);
(Accelerated) hypofractionation: with, typically, more than 2.5 Gy per fraction, typically 34 Gy delivered in 10 fractions or 40 Gy delivered in 15 fractions, ideally, over 2 weeks up to 4 weeks.
Stereotactic Ablative Body radiotherapy (SBAR) [also known as SBRT (stereotactic body radiotherapy)], which may be considered as an extreme hypofractionation regime: with typically 45-60 Gy delivered in 3 fractions, 48-50 Gy delivered in 4 fractions, or 50-55 Gy delivered in 5 fractions, typically over 2 weeks.

Therefore, in the above-mentioned clinical context, especially when fractionated radiation is used, wherein radioenhancers are repeatedly exposed to ionizing radiation over a period of at least 2 weeks, preferably 3 or 4 weeks, radioenhancer stability is a critical factor to ensure an optimal radioenhancement effect and an optimal benefit/risk ratio for the patient throughout the entire period of RT sessions.

Overall, there is an unmet need to provide stable radioenhancers that may fulfil dual or multi-function purposes as therapeutic agents. These compounds must present sustained efficacy and stability in vivo, especially when exposed to fractionated radiotherapy (RT) sessions, as typically delivered in humans whatever the type of ionizing radiation used (photon, protons, electrons, etc.) and/or the radiation technique used (3D-CRT, IMRT, etc.).

The inventors have identified a series of porous high-Z particles with surprisingly advantageous stability, which can advantageously be used as radioenhancers, particularly in the context of fractionated radiotherapy.

### DESCRIPTION OF THE INVENTION

It is the subject of the present invention to provide an effective radioenhancer with enhanced stability and efficacity, suitable for use in repeated RT sessions, that may also provide at least one other therapeutic function. The inventors herein provide such systems in the form of porous and carbon-free particles containing high-Z elements (i.e., the particle does not possess any chemical bonds involving a carbon element).

Without being bound by theory, the inventors attribute the enhanced stability of the inventive particles to the absence of the carbon element within the particle.

The inventors herein describe a porous, high-Z and carbon-free particle having internal pores, of longest dimension of at least 0.5 nm, as a new radioenhancer. This particle may comprise:
a. a high-Z metal phosphate, a high-Z metal oxo phosphate, a high-Z metal oxide or a high-Z mixed metal oxide, wherein the high-Z metal phosphate, high-Z metal oxo phosphate, high Z metal oxide or high-Z mixed metal oxide comprises at least one high-Z metal element, wherein the Z value of the at least one high-Z metal element is of at least 40; or
b. a plurality of high-Z metal phosphate layers, wherein the Z value of the high-Z metal element of each metal phosphate layer is of at least 40 and wherein each high-Z metal phosphate layer is connected to an adjacent high-Z metal phosphate layer via a carbon free linker;
   and optionally, a biocompatible surface coating, and wherein the particle is for use in altering or destroying target cancerous cells in a mammal when said cells are exposed to ionizing radiation.

The present invention also provides a porous, high-Z and carbon-free particle wherein at least part of the porous structure of the particle is occupied by at least one therapeutic moiety (also herein identified as a therapeutic agent). This at least one therapeutic moiety/agent is preferably for use in oncology. The therapeutic agent is typically selected from an immunotherapeutic agent, a cytotoxic agent, a targeted therapeutic agent, a photothermal agent, a photodynamic agent and any mixture thereof.

The inventors also describe a composition comprising the porous, high-Z and carbon-free particles, that may be coated with a biocompatible surface coating, and a pharmaceutically acceptable carrier, vehicle or support. The composition may comprise either type of the particles a., or b., or it may comprise a mixture of both.

In one embodiment of the invention, the composition comprising the particles is for use in altering or destroying target cancer cells in a mammal, particularly in a human, when said cells are exposed to ionizing radiation.

### FIGURES

FIG. 1. Schematic representation of the porous, high-Z and carbon-free particle of the invention according to claim 1. The schematic representations can be extended in each of the three dimensions to obtain the resulting porous, high-Z and carbon-free particle.

FIG. 1A shows two high-Z (Z≥40) metal connected to each other via phosphate groups, oxo phosphate groups, or oxide groups. In the case of metal oxides, the high-Z metal (M) may be the same (M) or different (M′), thus generating a mixed metal oxide.

FIG. 1B shows two high-Z (Z≥40) metal phosphate layers connected to each other via inorganic (i.e., carbon-free) linkers.

FIG. 2A: X-ray Powder Diffraction Pattern (XRD) of a sample of Example 1

FIG. 2B: Transmission Electron Microscopy (TEM) micrograph of a sample of Example 1. The scale is shown on the figure (as it is for all of the TEM micrographs).

FIG. 3A: XRD of Examples 1, 2 and 3

FIG. 3B: TEM of Example 2

FIG. 3C: TEM of Example 3

FIG. 4: TEM of Example 4

FIG. 5A: TEM of Example 5.

FIG. 5B: XRD of samples of Example 5, which have been incubated in Fetal Bovine Serum (FBS) to demonstrate product stability in an organic environment. The lowest diffractogram is before incubation, the middle diffractogram is after incubation in FBS for seven and a half (7.5) days, and the uppermost diffractogram is after incubation in FBS for fifteen (15) days.

FIG. 6A: XRD of Example 6

FIG. 6B: TEM of Example 6

FIG. 7A: TEM of Example 7

FIG. 7B: XRD of samples of Example 7, which have been incubated in Fetal Bovine Serum (FBS) to demonstrate product stability in an organic environment. The lowest diffractogram is before incubation, the middle diffractogram is after incubation in FBS for seven (7) days, and uppermost diffractogram is after incubation in FBS for fifteen (15) days.

FIG. 7C: XRD of samples of Example 7 which have been irradiated with X-rays to demonstrate product stability under irradiation. The lowest diffractogram is before irradiation, the middle diffractogram is after irradiation with 2 Gy, and the uppermost diffractogram is after irradiation with 20 Gy

FIG. 8: TEM of Example 8

FIG. 9A: TEM of Example 9

FIG. 9B: Stability in FBS of Example 9

FIG. 10A: WST1 efficacy assay with Example 1 at 650 μM

FIG. 10B: WST1 efficacy assay with Example 1 at 325 μM

FIG. 11A: WST1 efficacy assay with Example 4 at 1410 μM

FIG. 11B: WST1 efficacy assay with Example 4 at 705 μM

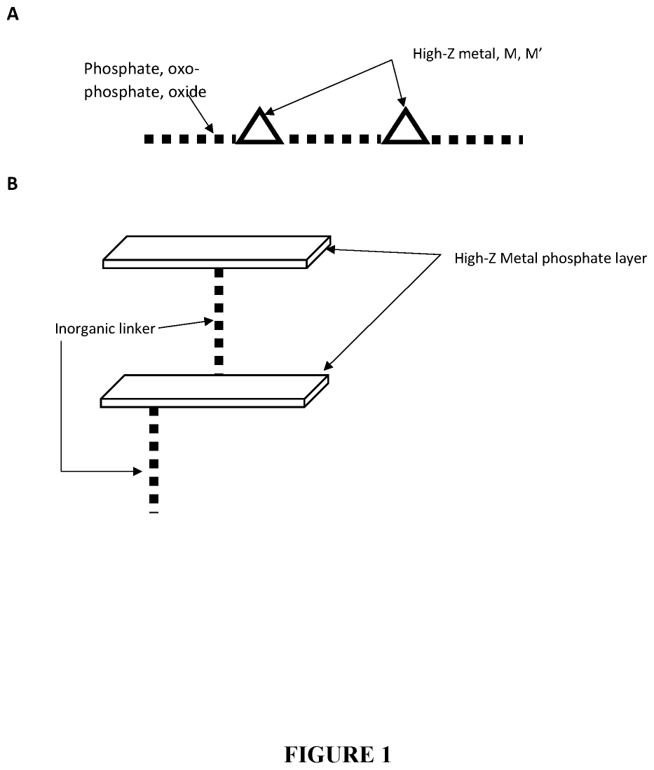

### DEFINITIONS

Metal-oxo clusters are broadly defined as polynuclear species (meaning containing two or more metal cations) with ligands of H₂O, OH and/or O₂ and are molecular. “Molecular” means they have a distinct chemical formula [see Polyoxometalates and Other Metal-Oxo Clusters in Nature. May Nyman Department of Chemistry, Oregon State University, Corvallis, OR, USA. Springer International Publishing Switzerland 2016 W. M. White (ed.), Encyclopedia of Geochemistry, DOI 10.1007/978-3-319-39193-9, 43-1]. A plurality of metal-oxo clusters means at least two metal-oxo clusters, preferably more than three, four, five, or ten, or fifteen or twenty. The final composition and the particle size will determine the total number of metal-oxo clusters comprised within the particle.

A high-Z (metal) element is an element of the Mendeleev's periodic table which has an atomic number value (i.e., a Z value) of at least 20, preferably above 25, even more preferably of at least 40, 45, 50, 55, 60, 61, 62, 63, 64, 65, 66, 67, 68, 69, 70, 71, 72, 73, 74, 75, 76, 77, 78, 79, 80 or 81.

A high-Z metal oxo-cluster is a metal oxo-cluster comprising at least one high-Z (metal) element.

Polyoxometalates, commonly known as POMs or POM clusters, are a subset of metal-oxo clusters. POM clusters comprise group 5 or 6 transition metals (V, Nb, Ta, Mo and W) in their highest oxidation states, linked by oxygen atoms.

Metal phosphates or metallophosphates are defined as purely inorganic materials composed of the binding of metal sites with phosphoric acid. Metallophosphates with rich 3D architectures, constitute materials having porosities that widely vary from small micropores to very large mesopores and macropores.

Metal phosphates or metallophosphates layers are 2-dimensional (2D) sheets made of metal phosphates.

An open pore (also named accessible pore), as opposed to a close pore, is a pore not totally enclosed by its walls and open to the surface, either directly or by interconnecting with other pores and therefore (in the present context) accessible to fluid. Unless specified otherwise, the term pore is to be understood as open pore in the context of the present invention.

A macropore is a pore of internal width greater than 50 nm.

A mesopore is a pore of internal width between 2 nm and 50 nm.

A micropore is a pore of internal width of less than 2 nm.

Porosity is the ratio of the volume of open pores and voids to the total volume occupied by the solid. Gas adsorption methods can be used to assess the porosity of the particle. Alternatively, Mercury Intrusion Porosimetry or small angle X-ray scattering (SAXS) methods can be used to assess the particle's porosity. High-resolution transmission electronic microscopy (HRTEM) may also be used to assess pore shape and/or pore size (also known as pore width or pore internal width).

The specific surface area of the porous, high-Z and carbon-free particle is herein defined as the BET surface area of the porous, high-Z and carbon-free particle and is typically measured using the Brunauer-Emmett-Teller (BET) gas (preferably nitrogen) adsorption method. In the context of the present invention, the BET surface area of the porous, high-Z and carbon-free particle is typically of at least 50 m²/g, preferably of at least 100 m²/g, at least 200 m²/g, at least 300 m²/g, at least 400 m²/g or at least 500 m²/g, when measured using Brunauer-Emmett-Teller (BET) gas (i.e., nitrogen) adsorption method.

In the context of the present invention, the term high-Z metal oxo-clusters directly connected/linked together corresponds to the formation of bonds between adjacent high-Z metal oxo-clusters, such as covalent, complexing (or coordination) bonds, and/or ionic bonds. Preferably, the connection corresponds to the formation of strong bonds between adjacent high-Z metal oxo-clusters, typically covalent and/or complexing bonds.

The terms “treatment” or “therapy” refer to both therapeutic and prophylactic or preventive treatment or measures that can significantly slow disease progression (for example, stop cancerous tumor growth) or increase/improve Progression Free Survival (PFS) or Overall Survival (OS), or cure a cancer (i.e., turn the patient into a cancer survivor, as further defined herein below).

Such a treatment or therapy is intended for a subject in need thereof, in particular, a mammal, preferably a human being, typically a human patient suffering from a malignant solid tumor.

In the art and in the context of the present invention, the terms “treatment having curative intent”, “curative treatment” or “curative therapy” refer to a treatment or therapy, in particular, a treatment comprising a radiotherapeutic step, offering to the subject to be treated a curative solution for treating the cancer(s) he/she is affected by, that is, for globally treating said subject [primary tumor(s) as well as corresponding metastatic lesion(s)].

As well known by the skilled person, the terms “palliative treatment” including in particular “palliative radiotherapy” are used for palliation of symptoms and are distinct from “radiotherapy”, i.e., radiotherapy delivered as curative treatment (also herein identified as “curative radiotherapy”). Indeed, palliative treatment is considered by the skilled person as an efficacious treatment for treating many symptoms induced by locally advanced or metastatic tumors, even for patients with short life expectancy.

In the context of the invention, a patient cured from its cancer is identified a “cancer survivor”. Globally, more than 33 million people are now counted as cancer survivors, and in resource-rich countries, such as the United States, extended survival means that more than 67% of patients survive more than 5 years and more than 25% of patients survive more than 15 years. Long-term cancer survivors (patients who survive more than 15 years) may be considered to be ‘cured’ of their cancer [Dirk De Ruysscher et al. Radiotherapy Toxicity. Nature Reviews, 2019, 5].

### DETAILED DESCRIPTION OF THE INVENTION

Inventors herein describe a porous, high-Z and carbon-free particle having internal pores of longest dimensions of at least 0.5 nm, as a new radioenhancer. This particle comprises:
a. a high-Z metal phosphate, a high-Z metal oxo phosphate, a high-Z metal oxide or a high-Z mixed metal oxide, wherein the high-Z metal phosphate, high-Z metal oxo phosphate, high Z metal oxide or high-Z mixed metal oxide comprises at least one high-Z metal element, wherein the Z value of the at least one high-Z metal element is of at least 40; or
b. a plurality of high-Z metal phosphate layers, wherein the Z value of the high-Z metal element of each metal phosphate layer is of at least 40 and wherein each high-Z metal phosphate layer is connected to an adjacent high-Z metal phosphate layer via a carbon free linker;
   and optionally, a biocompatible surface coating, and wherein the particle is for use in altering or destroying target cells, in particular target cancerous cells, in a mammal when said cells are exposed to ionizing radiation.

### Porous, High-Z and Carbon-Free Particle Size

In the spirit of the invention, the term “particle” refers to a product, in particular a synthetic product, with a size typically between about 1 nm and about 1000 nm, preferably between about 1 nm and about 500 nm.

The size of the particle can typically be measured by Electron Microscopy (EM) technics, such as transmission electron microscopy (TEM) or cryo-TEM, as well known by the skilled person. The size of at least 100 particles is typically measured (typically considering the particle's longest dimension) and the median size of the population of particles is reported as the size of the particles.

### A Porous, High-Z and Carbon-Free Particle Shape

As the shape of the particles can influence their “biocompatibility”, particles having a quite homogeneous shape are preferred. For pharmacokinetic reasons, particles being essentially spherical, round, or ovoid in shape are, thus preferred. Such a shape also favors the particles' interaction with, or uptake by cells.

### A Porous, High-Z and Carbon-Free Particle Composition

### High-Z Metal Phosphates, High-Z Metal Oxo Phosphates, High-Z Metal Oxides or High-Z Mixed Metal Oxides Forming Porous Frameworks

In in aspect of the invention, the porous, high-Z and carbon-free particle used as a radioenhancer in the context of the invention, comprises a high-Z metal phosphate, a high-Z metal oxo phosphate, a high-Z metal oxide or a high-Z mixed metal oxide, wherein the high-Z metal phosphate, high-Z metal oxo phosphate, high Z metal oxide or high-Z mixed metal oxide comprises at least one high-Z metal element, wherein the Z value of the at least one high-Z metal element is of at least 40.

According to an embodiment of this aspect of the invention, the high-Z metal is chosen from a lanthanide, tantalum (Ta), tin (Sn), zirconium (Zr), cerium (Ce), hafnium (Hf), tungsten (W), niobium (Nb), titanium (Ti) or rhenium (Re).

When the porous, high-Z and carbon-free particle, used as a radioenhancer in the context of the invention, comprises a high-Z metal phosphate, the metal phosphate composition may be typically MPO₄·xH₂O, wherein M is a metal element having a Z value of at least 40. Typically, M can be a lanthanide element, a zirconium (Zr) element or a hafnium (Hf) element. In a particular aspect, the porous, high-Z and carbon-free particle comprises a high-Z metal phosphate, wherein the metal phosphate is selected from a hafnium phosphate, a zirconium phosphate and a lanthanide phosphate.

A typical synthesis of zirconium phosphate with mesopores is described in Jose Jiménez-Jiménez et al. Surfactant-Assisted Synthesis of a Mesoporous Form of Zirconium Phosphate with Acidic Properties. Adv. Mater. 1998, 10, No. 10, 812-814. Example 8 herein describes the synthesis and characterization of a mesoporous zirconium phosphate according to the latter method. Example 1 details the synthesis and characterization of another mesoporous zirconium phosphate structure according to a protocol adapted from [Tarafdar, A., (2006). Synthesis of spherical mesostructured zirconium phosphate with acidic properties. Microporous and mesoporous materials, 95(1-3), 360-365.] Example 6 details the synthesis and characterization of the hafnium phosphate analog of Example 1. Example 7 details the synthesis and characterization of a porous hafnium phosphate structure.

When the porous, high-Z and carbon-free particle, used as a radioenhancer in the context of the invention, comprises a high-Z metal oxo phosphate, the metal oxo phosphate composition is typically M₂O(PO₄)₂·xH₂O, wherein M is a metal element, preferably a tetravalent metal cation, with a Z value of at least 40. Typically, M can be a lanthanide element, a zirconium (Zr) element or a hafnium (Hf) element. In a particular aspect, the porous, high-Z and carbon-free particle comprises a high-Z metal oxo phosphate, wherein the metal oxo phosphate is a hafnium oxo phosphate or a zirconium oxo phosphate. A typical synthesis of a uniformly arrayed zirconium oxo phosphate with micropores is described in Wooyong Um et al. Synthesis of nanoporous zirconium oxophosphate and application for removal of U(VI). Water Research 41 (2007) 3217-3226]. The nanoporous material exhibits high thermal stability.

Of note, the chemistry of zirconium cation and hafnium cation in aqueous solution is similar [C. F. Baes and R. S. Mesmer: The Hydrolysis of Cations. John Wiley & Sons, New York, London, Sydney, Toronto 1976]. Therefore, the hafnium cations can easily substitute the zirconium cations in the herein above-described syntheses.

According to an embodiment of the invention, the high-Z and carbon-free particle comprises Zr phosphate, and is obtainable by precipitation of a zirconium salt complex and a hydrogen phosphate salt in a basic medium, followed by a step of calcination or washing with an organic solvent, like ethanol. Typically, according to this embodiment of the invention, the internal pore diameter of the porous high-Z and carbon-free particle is approximately 2 nm.

According to one embodiment of the invention, the high-Z and carbon-free particle comprises hafnium phosphate, and is obtainable by a process that includes a step of precipitation of Hf(SO₄)₂, and a step of sulfate ions replacement with phosphate ions, and a calcination step or a step of washing with ethanol. Typically, according to this embodiment of the invention, the internal pore diameter of the porous high-Z and carbon-free particle is approximately 2 nm. When the porous, high-Z and carbon-free particle, used as a radioenhancer in the context of the invention, comprises a high-Z metal oxide or mixed metal oxide, the metal oxide or mixed metal oxide composition is typically MₓO_{y}, or MₓM′_{z}O_{y}, wherein M and M′ are metal elements chosen independently and M or M′ has a Z value of at least 40. Typically, M or M′ can be a lanthanide element, a zirconium (Zr) element or hafnium (Hf) element. In a typical aspect, the porous metal oxide or the porous mixed metal oxide is selected from Nb₂O₅, Ta₂O₅, WO₃, HfO₂, SnO₂, ZrTiO₄ and ZrW₂O₈. A typical synthesis of these metal oxides or mixed metal oxides with mesopores is described in Peidong Yang et al. (Generalized syntheses of large-pore mesoporous metal oxides with semicrystalline frameworks. Nature volume 396, pages 152-155(1998)). Descriptions of the synthesis and characterization of two different porous hafnium oxide structures are detailed in Examples 4 and 5, respectively.

According to one embodiment of the invention, the high-Z and carbon-free particle comprises a high-Z metal oxide or mixed metal oxide of respective composition MₓO_{y}, and MₓM′_{z}O_{y}, wherein M and M′ are metal elements chosen independently from a lanthanide element, zirconium (Zr) and hafnium (Hf).

According to one embodiment of the invention, the high-Z and carbon-free particle comprises hafnium oxide and is obtainable by a process including a hydrolysis/condensation step of HfCl₄, followed by a calcination step, or a washing step. Typically, the internal pore diameter of said porous high-Z and carbon-free particle is approximately 5 nm.

### High-Z Metal Phosphates Layers Forming Porous Frameworks

In another aspect of the invention, the porous, high-Z and carbon-free particle, used as a radioenhancer in the context of the invention, comprises a plurality of high-Z metal phosphate layers, wherein the Z value of the high-Z metal element of each metal phosphate layer is of at least 40 and wherein each high-Z metal phosphate layer is connected to an adjacent high-Z metal phosphate layer via a carbon-free linker.

According to one embodiment of this aspect of the invention, the high-Z metal is chosen from a lanthanide, tantalum (Ta), tin (Sn), zirconium (Zr), cerium (Ce), hafnium (Hf), tungsten (W), niobium (Nb), titanium (Ti) or Rhenium (Re).

In a particular aspect of the invention, the metal phosphate layers of the porous, high-Z and carbon-free particle of the invention, are hafnium phosphate layers, zirconium phosphate layers or a mixture thereof.

Examples of high-Z metal phosphate layers are the Zr(HPO₄)₂·H₂O (zirconium phosphate) or Hf(HPO₄)₂·H₂O (hafnium phosphate) 2D sheets. Typical synthesis of high-Z metal (IV) pillared layered phosphates are described in Pascual Olivera-Pastor et al. Nanostructured Inorganically Pillared Layered Metal (IV) Phosphates. Chem. Mater. 1996, 8, 1758-1769].

Generally, inorganic linkers act as intercalating agents between the phosphate layers and create the particle's porosity. Inorganic linkers that may be employed are typically metal oxo-clusters including POMs, borazocine (B₄N₄H₈), Se₆, dinitrogen units, nitrogen-based polymers (such as polydiazenediyl (polyacetylene-like) nitrogen chains) or inorganic nanoparticles, etc. The skilled person knows how to integrate the inorganic linkers between the phosphate layers, etc.

Descriptions of the synthesis and characterization of two different products according to these embodiments of the invention are given in Example 2 (zirconium oxide introduced into zirconium phosphate layered structure) and Example 3 (hafnium oxide introduced into zirconium phosphate layered structure), wherein, the Zr phosphate product of Example 1 is used as the starting material.

### Pore Size (or Pore Width)

In the context of the present invention, the porous, high-Z and carbon-free particles present a porosity defined by the presence of pores of longest dimension (also named pore size or pore width) of at least 0.5 nm, preferably of at least 1 nm and typically less than 100 nm, for example between 0.5-50 nm. The high-Z and carbon-free particles may be macroporous, mesoporous or microporous, i.e., they may contain macropores (pores of internal width greater than 50 nm), mesopores (pores of internal width between 2 nm and 50 nm) or micropores (pores of internal width of less than 2 nm). The mesoporous material preferably has a pore of internal width between 2 and 30 nm, more preferably between 2 and 20 nm, and even more preferably, between 2 and 14 nm. Typically, the porous particles have a uniform pore size throughout the bulk for a given batch of synthetized material.

When the porous particles contain at least one therapeutic agent, the porous particles are typically selected so that their pores' size fits with the dimension of the at least one therapeutic agent (i.e., the pore size is sufficient to trap the therapeutic agent). For example, the pore size in a material to trap a small molecule drug with a molecular weight typically less than 500 Da will be smaller than a pore to trap a protein (for example Pembrolizumab having a total buried surface area of approximately 200 nm²).

The pore shape can be any geometrical form, such as a cube, a diamond, a rectangle, a cylinder, or a sphere. In one embodiment of the invention, the pores of the particle are cubic shape pores, rectangular shape pores and/or diamond shape pores and the pores' size (i.e., also defined as the pores' longest dimension) in this context, reflect the diagonal of the cube, of the rectangle or of the diamond pore respectively. In a preferred embodiment of the invention, the pores of the particle are cylindrical or spherical, and the pores' size (i.e., also defined as the pores' longest dimension) in this context, reflects the radius of the cylinder, or of the spherical pore. When the pores of the particle have no well-defined geometries, the longest pores' size corresponds to the longest distance between two opposite internal walls of the pores. In the context of the present invention, the longest pores' size reflects the internal width of the pores.

The particles can contain micropores, mesopores and/or macropores. In a preferred embodiment, the pores of the particles are mesopores.

### Optional Biocompatible Coating

In a particular aspect of the description, each porous, high-Z and carbon-free particle of the present invention further comprises a biocompatible surface coating.

In a preferred aspect, each porous, high-Z and carbon-free particle used in the context of the present invention can be coated with a biocompatible material, preferably, with an agent exhibiting a stealth property. Indeed, when the porous, high-Z and carbon-free particles of the present invention are administered to a subject via the intravenous (IV) route, a biocompatible coating with an agent exhibiting a stealth property is particularly advantageous to optimize the biodistribution of the porous, high-Z and carbon-free particles. Such coating is responsible for the so called “stealth property” of the porous, high-Z and carbon-free particles. The agent exhibiting stealth properties may be an agent displaying a steric group. Such a group may be selected for example from polyethylene glycol (PEG); polyethylenoxide; polyvinylalcohol; polyacrylate; polyacrylamide (poly(N-isopropylacrylamide)); polycarbamide; a biopolymer; a polysaccharide such as for example dextran, xylan and cellulose; collagen; and a zwitterionic compound such as for example polysulfobetain.

In another preferred aspect, each of the porous, high-Z and carbon-free particles can be coated with an agent allowing non-specific interaction with a biological target. Such an agent can typically bring a positive or a negative charge on the porous, high-Z and carbon-free particle's surface. This charge can be easily determined by zeta potential measurements, typically performed on porous, high-Z and carbon-free particles suspensions the concentration of which vary between 0.2 and 10 g/L, the particles being suspended in an aqueous medium with a pH comprised between 6 and 8.

An agent forming a positive charge on the porous, high-Z and carbon-free particle's surface can be for example aminopropyltriethoxisilane or polylysine. An agent forming a negative charge on the porous, high-Z and carbon-free particle's surface can be for example a phosphate (for example a polyphosphate, a metaphosphate, a pyrophosphate, etc.), a carboxylate (for example citrate or dicarboxylic acid, in particular succinic acid) or a sulphate.

A full biocompatible coating of the porous, high-Z and carbon-free particle may be advantageous, in particular for an intravenous (IV) administration in the human patient, in order to avoid interaction of the particle's surface with any recognition element (macrophage, opsonins, etc.).

Advantageously, the coating ensures or improves the biocompatibility of the particles in vivo, and facilitates an optional functionalization thereof (for example with spacer molecules, biocompatible polymers, targeting agents, proteins, etc.).

### Optional Targeting

A particular porous, high-Z and carbon-free particle as herein described can further comprise a targeting agent associated with the said carbon free particle. A targeting agent typically recognizes an element present on a target cell, typically on a cancer cell. Such a targeting agent typically acts once the porous, high-Z and carbon-free particles are accumulated on the target site, typically on the tumor site. The targeting agent can be any biological or chemical structure displaying affinity for molecules present in the mammalian, in particular, the human, body. For instance, it can be a peptide, oligopeptide or polypeptide, a protein, a nucleic acid (such as, for example DNA, RNA, SiRNA, tRNA, miRNA, etc.), a hormone, a vitamin, an enzyme, the ligand of a molecule expressed by a pathological cell, in particular the ligand of a tumor antigen, hormone receptor, cytokine receptor or growth factor receptor. Said targeting agent can be, for example, selected in the group consisting in LHRH, EGF, a folate, anti-B—FN antibody, E-selectin/P-selectin, anti-IL-2Ra antibody and GHRH.

According to one embodiment of the invention the high-Z and carbon-free particle additionally comprises a targeting agent that recognizes an element present on a cancer cell and is chosen from peptide, oligopeptide, a protein, a nucleic acid (such as for example DNA, RNA, SiRNA, tRNA, miRNA, etc.), a hormone, a vitamin, or the ligand of any of a tumor antigen hormone receptor, cytokine receptor or growth factor receptor.

### Therapeutic Agent/Moiety

The present invention also provides a porous, high-Z and carbon-free particle wherein at least part of the porous structure of the particle is occupied by at least one therapeutic moiety (also herein identified as a therapeutic agent). The at least one therapeutic moiety is preferably for use in oncology. The therapeutic agent is typically selected from an immunotherapeutic agent, a cytotoxic agent, a targeted therapeutic agent, a photothermal agent, a photodynamic agent and any mixture thereof.

The immunotherapeutic agent can typically be any molecule, drug, oncolytic virus, DNA-based vaccine, peptide-based vaccine, toll-like receptor agonist, or any combination thereof, capable of boosting the immune system of a subject in need thereof, and recognized as such by the skilled person. The molecule or drug can for example be selected from a monoclonal antibody, a cytokine, and a combination thereof.

The drug can typically be an indoleamine 2,3-dioxygenase (IDO) inhibitor such as 1-methyl-D-tryptophan.

In a preferred embodiment, the monoclonal antibody inhibits the CTLA-4 molecule or the interaction between PD-1 and its ligands. The monoclonocal antibody is advantageously selected from anti-CTLA-4, anti-PD-1, anti-PD-L1, anti-PD-L2. The monoclonal antibody can for example be selected from ipilimumab, tremelimumab, nivolumab, prembolizumab, pidilizumab and lambrolizumab.

In another preferred embodiment, the monoclonal antibody enhances CD27 signaling, CD137 signaling, OX-40 signaling, GITR signaling and/or MHCII signaling, and/or activate CD40. The monoclonal antibody can for example be selected from dacetuzumab, lucatumumab, and urelumab.

In a further embodiment, the monoclonal antibody inhibits TGF-β signaling or KIR signaling. The monoclonal antibody can for example be selected from fresolimumab and lirilumab.

The cytokine can be advantageously selected from the granulocyte-macrophage colony stimulating factor (GM-CSF), a fins-related tyrosine kinase 3 ligand (FLT3L), IFN-α, IFN-α2b, IFNγ, IL2, IL-7, IL-10 and IL-15.

In another preferred embodiment, the immunotherapeutic agent is an immunocytokine, for example the immunocytokine L19-IL2.

The toll-like receptor agonist is advantageously selected from a TLR 2/4 agonist, a TRL 7 agonist, a TRL 7/8 agonist and a TRL 9 agonist. The toll-like receptor agonist can for example be selected from imiquimod, bacillus Calmette-Guérin and monophosphoryl lipid A.

A preferred combination of immunotherapeutic agents can be for example selected from a cytokine, a monoclonal antibody, a Toll-like receptor agonist and a peptide-based vaccine.

The “cytotoxic” or “cytotoxic drug” (typically used in the context of chemotherapy) is a drug that may be used to destroy cancer cells by typically inhibiting cell division and in this way causing cancer cells to die. Cytotoxic drugs typically belongs to the alkylating drugs family (e.g. cyclophosphamide), the anthracyclines and other cytotoxic antibiotics family (e.g. bleomycin, doxorubicin, or mitoxantrone), the antimetabolites family (e.g. fluorouracil, gemcitabine), the vinca alkaloids family (e.g. vincristine), the protein kinase inhibitors family (e.g. dasatinib, erlotinib, everolimus, imatinib, nilotinib, sunitinib), etoposide, amsacrine, bexarotene, bortezomib, carboplatin, cisplatin, crisantaspase, dacarbazine, docetaxel, hydroxycarbamide (hydroxyurea), irinotecan, oxaliplatin, paclitaxel, pentostatin, procarbazine, temozolomide, topotecan or tretinoin.

The targeted therapeutic agent is any agent that offer a way to develop very specific treatments while concomitantly resulting in little to no off-target toxicity. Typical classes of targeted therapeutic agents are antibodies (such as cetuximab or trastuzumab), antibody fragments, antisense oligodeoxynucleotides, fusion proteins and antibody-drug conjugates.

A photothermal agent is, typically, an agent that produces an artificial elevation of the tissue temperature surrounding this agent upon stimulation by the proper energy (e.g., near infrared light). Typical photothermal agents are inorganic-based nanoparticles such as metal-based nanoparticles (e.g., gold (Au) nanoparticles). Alternatively, photothermal agents can be made from organic agents including cyanine-based agents (e.g., indocyanine green), diketopyrrolopyrrole-based agents, croconaine-based agents, or porphyrin-based agents.

A photodynamic agent is typically a photosensitizing agent that produces cytotoxic reactive oxygens upon exposure to the appropriate light in presence of oxygen. Typical examples of photosensitizing agents are inorganic-based nanoparticles such as titanium oxide nanoparticles or quantum dots, or organic-based agents such as photofrin, temoporfin, motexafin lutetium, palladium bacteriopheophorbide, verteporfin or talaporfin.

### Cancer Indications

The porous, high-Z and carbon-free particles herein described are typically for use in altering or destroying target cells in a mammal, in particular, a human, when said cells are exposed to ionizing radiation.

The herein described invention can be applied to any target cancerous cells, in particular tumor cells, typically in a human patient suffering from a solid tumoral cancer.

Advantageously, the herein described carbon-free, inorganic, porous structures are efficacious as radioenhancers for use in radiotherapy for the treatment of cancer. Furthermore, said structures are structurally and chemically stable, which is of utmost importance in cases where the patient receives several radiation doses over a period of time (typically, weeks).

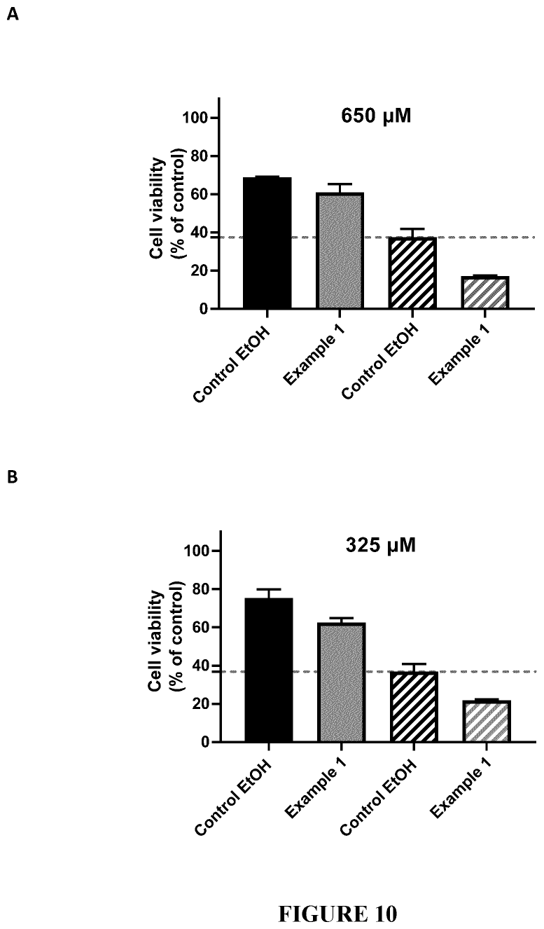

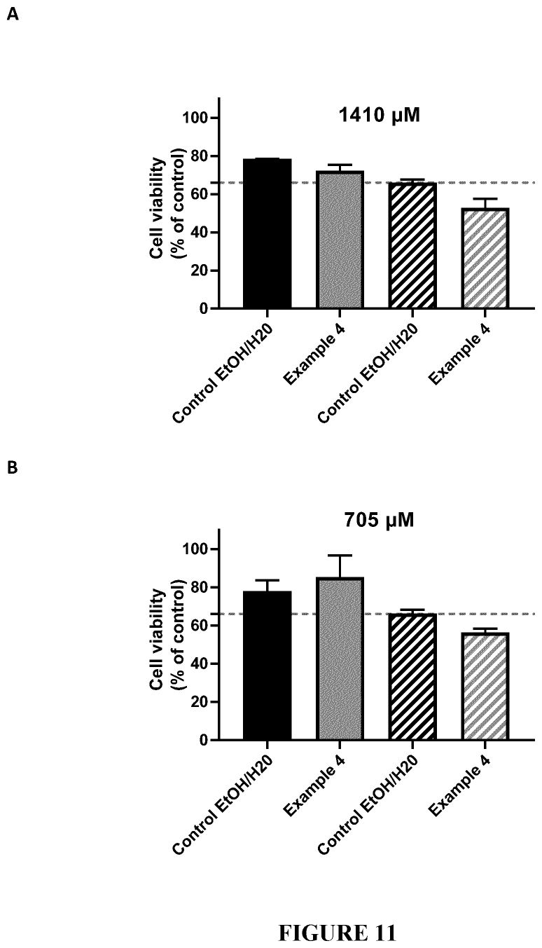

The in vitro efficacy of the particles of Example 1 and Example 4 was tested using a cell viability assay with a CT26 cell line. The results are described in Example 10, and shown in FIGS. 10 and 11. The in vitro test records the radioenhancing effect, in the presence of X-rays, of the claimed porous structures. The decrease in cell viability is measured after irradiation, in the presence of the claimed products versus the control (vehicle). The table below summaries the values obtained. Cell viability was expressed as % of the control (non-irradiated, untreated cells).

TABLE 1
results from the cell viability assay showing the radioenhancing
effect of examples of the claimed products.

| | Cell viability % after |
|---|---|
| Sample | irradiation |
| Control Ex 1 | 37 |
| Ex. 1_350 μM | 22 |
| Ex. 1_650 μM | 17 |
| Control Ex 4 | 66 |
| Ex. 4_705 μM | 56 |
| Ex. 4_1410 μM | 53 |

For both samples tested, the radioenhancement effect is observed. A higher rate of cell killing is observed in the samples that were incubated with the inventive products, compared to the control samples.

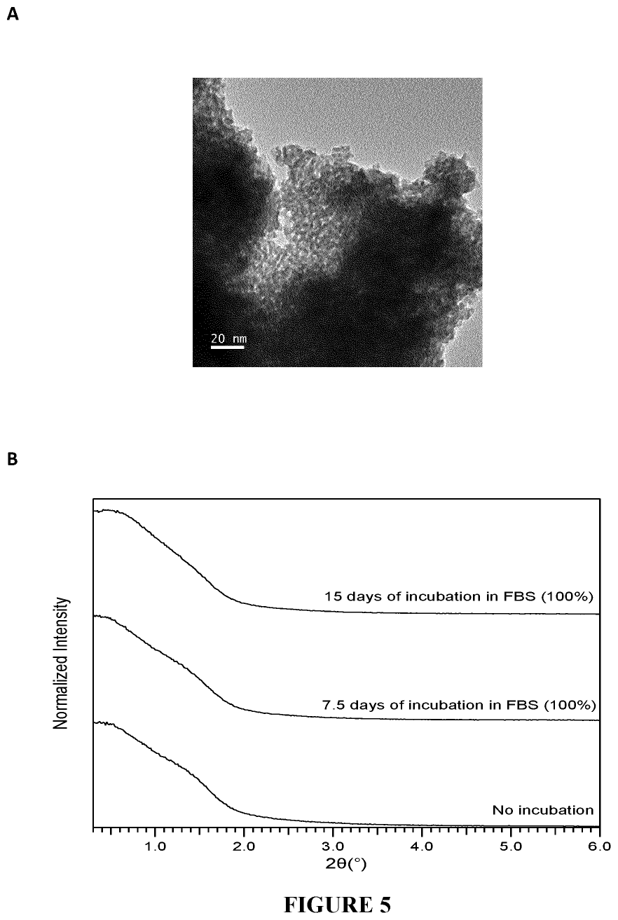

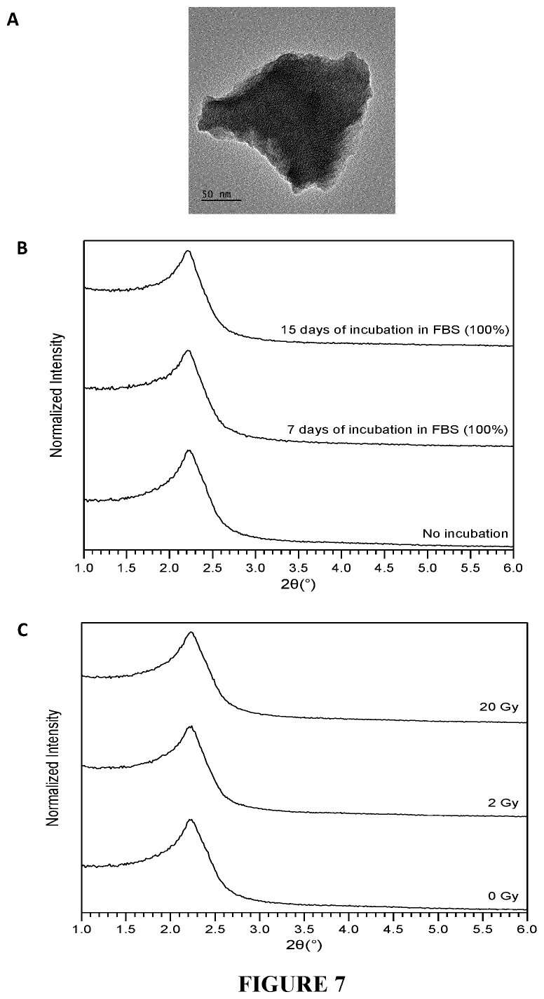

The Inventors also demonstrate herein that the claimed products are stable in a biological-like environment for at least 15 days, maintaining their porous structure (see Examples 5 and 7 and FIGS. 5B and 7B showing the product stability in fetal bovine serum (FBS)). This structural stability is also observed after irradiation with X-rays (see Example 7 and FIG. 7C). Thus, the claimed products are stable to X-rays and are also stable in an organic medium, such as FBS. This represents a major advantage in the therapeutic scheme of fractionated radiation, wherein the radioenhancer is repeatedly exposed to ionizing radiation over a period of at least two weeks, preferably, three or four weeks. Advantageously, the claimed products allow the radioenhancement effect to be maintained throughout all the RT sessions. Consequently, the treatment has a constant benefit/risk ratio for the patient, throughout the entire RT treatment period.

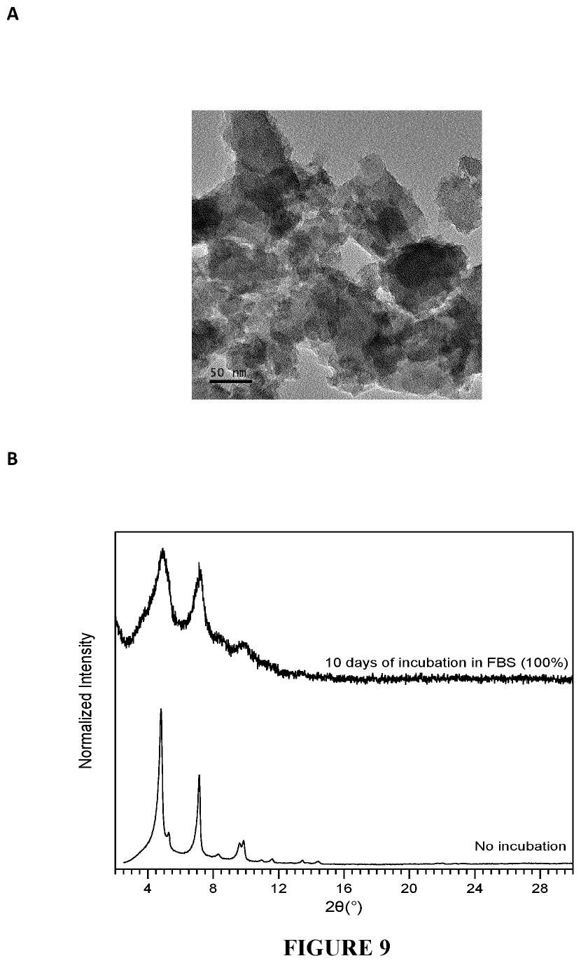

This structural stability presents an advantage with respect to typical metal organic frameworks discussed above. Indeed, the Applicant has shown that, a representative metal organic framework (MOF) structure (described in Reference Example 9) lacks structural stability when incubated in fetal bovine serum (FBS) for ten days (FIG. 9B). The Applicant has also found that the latter product was not efficacious as a radioenhancer, when tested in vitro.

Thus, in view of the demonstrated enhanced stability, the herein claimed porous high-Z carbon-free particles are advantageous for use as radioenhancers, in the treatment of cancer, with respect to organic based radioenhancers, for example MOF-based radioenhancers, which demonstrate poor stability upon irradiation (most likely, due to the oxidation of the organic linkers).

The cancer is for example a skin cancer, in particular a malignant neoplasm associated to AIDS; a melanoma; a squamous cancer; a central nervous system cancer such as for example a brain, cerebellum, pituitary, spinal cord, brainstem, eye or orbit cancer; a head and neck cancer, a lung cancer, a breast cancer, a gastrointestinal cancer such as a liver or a hepatobiliary tract cancer, a colon, a rectum and/or an anal cancer, a stomach cancer, a pancreas cancer, an esophagus cancer; a male genitourinary cancer such as for example a prostate, testis, penis or urethra cancer; a gynecologic cancer such as for example a uterine cervix, endometrium, ovary, fallopian tube, vagina and/or vulvar cancer; an adrenal and/or retroperitoneal cancer; a sarcoma of bone and soft tissue regardless its localization; or a pediatric cancer such as for example a Wilm's cancer, a neuroblastoma, a central nervous system cancer or a Ewing's sarcoma.

In a particular aspect of the invention, the target cancerous cells belong to a solid malignant tumor selected from a skin cancer, a central nervous system cancer, a head and neck cancer, a lung cancer, a liver cancer, a breast cancer, a gastrointestinal cancer, a male genitourinary cancer, a gynecologic cancer, an adrenal and/or retroperitoneal cancer, a sarcoma and a pediatric cancer.

### Composition

The inventors also describe a composition comprising a) porous, high-Z and carbon-free particles, wherein each particle has internal pores of longest dimension of at least 0.5 nm, for example, of between 0.5-50 nm, and wherein each particle comprises:
a. a high-Z metal phosphate, a high-Z metal oxo phosphate, a high-Z metal oxide or a high-Z mixed metal oxide, wherein the high-Z metal phosphate, high-Z metal oxo phosphate, high Z metal oxide or high-Z mixed metal oxide comprises at least one high-Z metal element, wherein the Z value of the at least one high-Z metal element is of at least 40; or
b. a plurality of high-Z metal phosphate layers, wherein the Z value of the high-Z metal element of each metal phosphate layer is of at least 40 and wherein each high-Z metal phosphate layer is connected to an adjacent high-Z metal phosphate layer via a carbon free linker;
   and, optionally a biocompatible surface coating, and a pharmaceutically acceptable carrier, vehicle or support.

Thus, according to an aspect of the invention, the composition may comprise any one of the particles a., or b., or, it may comprise a mixture of both.

The pharmaceutical composition herein described is, in a preferred aspect herein described, for use for preventing or treating cancer in a human patient.

In one aspect of the invention, the composition containing the particles is for use in altering or destroying target cancer cells in a mammal, particularly in a human, when said cells are exposed to ionizing radiation.

The composition may be in the form of a solid, liquid (typically porous, high-Z and carbon-free particles in suspension), aerosol, gel, paste, and the like. Preferred compositions are in a liquid or a gel form. Particularly preferred compositions are in liquid form.

The carrier which is employed can be any classical pharmaceutical support for the skilled person, such as for example a saline, isotonic, sterile, buffered solution, or a non-aqueous vehicle solution and the like.

The pharmaceutical composition herein described, may comprise a vehicle or support chosen from a liposome, viral vector, viral-like particle, albumin containing carrier, inorganic polymer and organic polymer known to the skilled person. The vehicle or support may also be any other suitable vehicles or supports known to the skilled person.

The composition can also comprise stabilizers, surfactants, polymers and the like. It can be formulated for example as an ampoule, aerosol, bottle, tablet, capsule, by using techniques of pharmaceutical formulation known by the skilled person.

Generally, the composition, in liquid or gel form, comprises between about 0.05 g/L and about 450 g/L of porous, high-Z and carbon-free particles, for example between about 0.05 g/L and about 250 g/L of porous, high-Z and carbon-free particles, preferably at least about 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 85 g/L, 90 g/L, 95 g/L, 100 g/L, 150 g/L, 200 g/L, 250 g/L, 300 g/L, 350 g/L, or 400 g/L of porous, high-Z and carbon-free particles.

The concentration of particles in the composition can be measured by dry extract. A dry extract is ideally measured following a drying step of the suspension comprising the particles in a drying oven.

### Administration Route

The porous, high-Z and carbon-free particles of the invention can be administered to the subject using different possible routes such as local (intra-tumoral (IT)), intra-arterial (IA), subcutaneous, intravenous (IV), intra-dermic, airways (inhalation), intraperitoneal, intramuscular, intra-articular, intra-thecal, intra-ocular or oral route (per os), preferably using IT, IV or IA. Preferably, the porous, high-Z and carbon-free particles of the invention are administered to the subject using an intra-tumoral (IT) route, for example by intra-tumoral injection.

Repeated injections or administrations of porous, high-Z and carbon-free particles can be performed, when appropriate.

### Radiotherapy Sources

In a typical aspect herein described, porous, high-Z and carbon-free particles are to be administered to the subject to be treated and said subject is then to be exposed to ionizing radiation. This ionizing radiation is typically selected from X-rays, gamma-rays, electrons and protons. Preferred ionizing radiations are X-rays.

In a preferred aspect, the particles of the invention, or the subject who has been administrated with the particles of the invention, is to be exposed to ionizing radiation.

RT comprises multiple different treatment modalities, including external beam therapy (encompassing photons, electrons, protons and other particles) and internal/surface treatment (brachytherapy and radiopharmaceuticals). In a preferred aspect herein described, ionizing radiation is selected from X-rays, gamma-rays, electrons and protons.

As indicated herein above, appropriate radiation is preferably ionizing radiation and can advantageously be selected from the group consisting of X-Rays, gamma-Rays, electron beams (electrons), ion beams (such as protons) and radioactive isotopes or radioisotopes emissions. X-Rays are particularly preferred ionizing radiation.

Ionizing radiation are typically of about 2 KeV to about 25 000 KeV, in particular of about 2 KeV to about 6000 KeV (i.e., 6 MeV) (LINAC source).

In general, and in a non-restrictive manner, the following X-Rays can be applied in different circumstances to excite the herein described particles:
superficial X-Rays of 2 to 50 keV: to excite nanoparticles near the skin surface (penetration of a few millimeters);
X-Rays of 50 to 150 keV: in diagnostic but also in therapy;
X-Rays (ortho voltage) of 200 to 500 keV which can penetrate a biological tissue thickness of 6 cm;
X-Rays (mega voltage) of 1000 keV to 25 000 keV.

Radioactive isotopes can alternatively be used as ionizing radiation (typically in the context of curie therapy or brachytherapy). In particular, Iodine 1-125 (t1/2=60.1 days), Palladium Pd-103 (t1/2=17 days), Cesium Cs-137, Strontium Sr-89 (t1/2=50.5 days), Samarium Sm-153 (t1/2=46.3 hours), and Iridium Ir-192, can advantageously be used.

Electron beams may also be used as ionizing radiation and have an energy typically comprised between 4 MeV and 25 MeV.

In a particular aspect, a specific monochromatic irradiation source can be used for selectively generating X-rays radiation at energy close to, or corresponding to, the desired X-ray absorption edge of the high-Z elements of the particles selected for use in the context of the invention.

Preferentially, ionizing radiations are X-rays obtained from Linear Accelerator (LINAC) or are protons.

In the context of the present invention, cells are exposed to ionizing radiation in the context of a radiotherapy regimen which is selected from a conventional fractionation regimen, an hyperfractionation regimen, an (accelerated) hypofractionation regimen and a stereotactic ablative body radiotherapy (SBAR) regimen.

The radiotherapy protocol for each patient is typically defined by the clinical team according to the patient cancer characteristics, the patient clinical staging, the patient health status, etc. The radiotherapy protocol may therefore correspond for example, to a definitive treatment, a palliative treatment, a concurrent or sequential systemic therapy/radiotherapy, a pre-operative radiotherapy, a post-operative radiotherapy.

In a preferred embodiment, the radiotherapy regimen (also named radiotherapy schedule) is a conventional fractionation comprising or consisting in, typically, from 1.6 Gy up to 2.25 Gy per fraction (for example 1.6, 1.8, 2.0, 2.12, 2.25 Gy per fraction), daily (Monday to Friday, i.e., five consecutive days per week), over 5 weeks up to 7 weeks;

In another embodiment, the radiotherapy regimen is a hyperfractionation comprising or consisting in, typically, 1.2 Gy per fraction, twice daily, over 7 weeks (for example from 79.2 Gy up to 81.6 Gy over 7 weeks, twice daily);

In another embodiment, the radiotherapy regimen is an (accelerated) hypofractionation comprising or consisting in, typically, more than 2.5 Gy per fraction, typically 34 Gy delivered in 10 fractions or 40 Gy delivered in 15 fractions, ideally over 2 weeks up to 4 weeks;

In another embodiment, the radiotherapy regimen is a stereotactic ablative body radiotherapy (SBAR) comprising or consisting in, typically, between 45 and 60 Gy delivered in 3 fractions, between 48 and 50 Gy delivered in 4 fractions, between 50 and 55 Gy delivered in 5 fractions, typically over 2 weeks.

It is of particular relevance to select the particles of the invention, or a composition comprising said particles, for use in the above-mentioned clinical context, especially when fractionated radiotherapy is used. This is because, in fractionated radiotherapy, the radioenhancers are repeatedly exposed to ionizing radiation over a period of typically at least 2 weeks, preferably 3 or 4 weeks. The porous, high-Z and carbon-free particles described herein are especially suitable for use when the fractionated regimen is given over more than ten days, or more than two weeks, or more than three weeks, or even more than seven weeks.

The particles of the present invention have the required stability to ensure an optimal radioenhancement effect, thus ensuring an optimal benefit/risk ratio for the patient throughout the entire period of RT sessions.

The examples which follow illustrate the invention without limiting the scope thereof.

### EXAMPLES

### Example 1: Porous Zirconium Phosphate

### Synthesis:

Porous zirconium phosphate was obtained by precipitation using a zirconium carbonate complex and diammonium hydrogen phosphate in a basic medium. The protocol was adapted from Tarafdar, A., (2006). Synthesis of spherical mesostructured zirconium phosphate with acidic properties. Microporous and mesoporous materials, 95(1-3), 360-365.

1.8 g of ZrOCl₂·8H₂O (Supelco®, by Merck, Darmstadt, Germany) was dissolved in 100 mL of distilled water. 3.8 g of (NH₄)₂CO₃ (Sigma-Aldrich®, by Merck) was added under stirring until a clear solution was obtained. Afterward, 1.5 g of (NH₄)₂HPO₄ (Supelco®,) was added and dissolved in the solution. 0.6 g of (1-Tetradecyl)trimethylammonium bromide (TTBr from Alfa Aesar, Haverhill, Massachusetts, USA) was added to the solution with continuous stirring. The resultant clear solution was kept in an oven at 80° C. for 3 days, in a closed polypropylene tube for complete precipitation, and then, aged in a Teflon autoclave at 90° C. for two days and then at 120° C. for 24 h. The sample was cooled and then washed three times with distilled water. The solid obtained was dried at 65° C. overnight and calcined at 540° C. for 6 h.

### Characterization:

The zirconium and phosphorus content of the powder was determined using Inductively Coupled Plasma-Optical Emission Spectrometry (ICP-OES). The ratio Zr/P was 0.7.

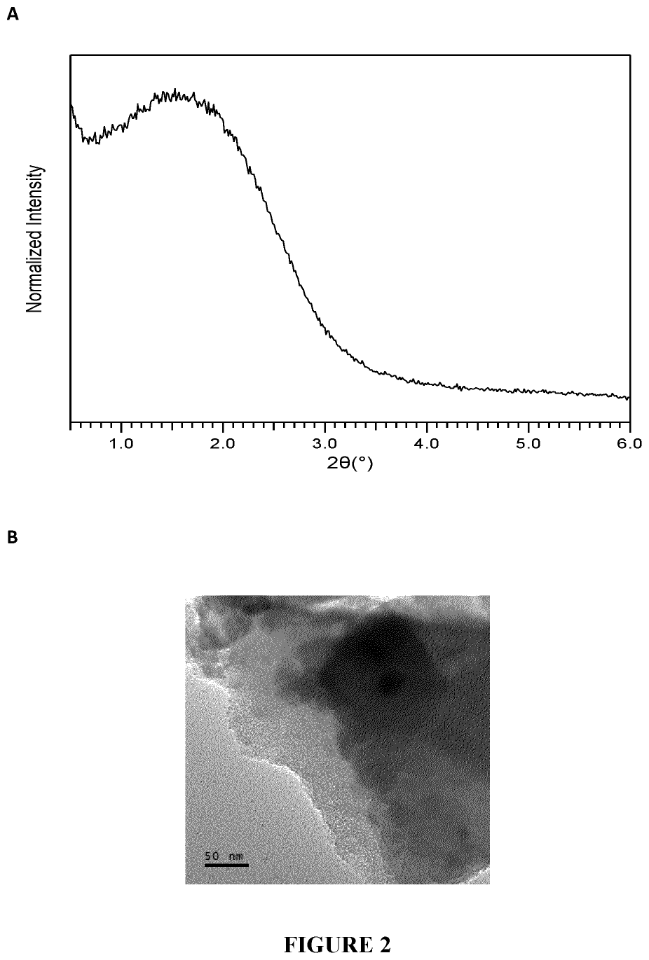

The X-Ray Diffraction (XRD) pattern of the calcined powder showed a single broad diffraction peak in the low angle region (2 θ=1.6°) with a corresponding d₁₀₀ value of 5.4 nm (FIG. 2A).

The Brunauer, Emmett and Teller (BET) specific surface area for all samples was estimated by nitrogen adsorption/desorption measurement at 77 K in a relative pressure range of 0.0001-1.0. For Example 1, the specific surface area of the powder was measured to be 149 m²/g and the pore diameter is 2.0 nm, lying between microporosity and mesoporosity.

The Transmission Electron Microscopy (TEM) micrograph (FIG. 2B) at high resolution confirmed the porous structure found by XRD and BET.

The InfraRed spectra (IR) results are detailed in Table 2 below. The presence of Zr—O stretching deformation, P—O stretching vibration and P—O—H stretching vibration confirmed the metal phosphate composition.

TABLE 2

| | | Reference | |
|---|---|---|---|
| | Wavenumber | Wavenumber | |
| IR Band | (cm⁻¹) | (cm⁻¹) | References |
| P—O—H | 2368 | 2350 | Um, W. (2007). Synthesis of nanoporous zirconium |
| vibration | | | oxophosphate and application for removal of U (VI). Water |
| | | | Research, 41(15), 3217-3226. |
| P—OH | 1240 | 1250 | Xiao, J. (1999). Preparation and properties of zirconia- |
| vibration | | | pillared zirconium phosphate and |
| | | | phenylphosphonate. Applied Catalysis A: General, 181(2), |
| | | | 313-322. |
| P—O | 1048 | 1042 | Hajipour, A. R. (2014). Synthesis and characterization of |
| vibration | | | hexagonal zirconium phosphate nanoparticles. Materials |
| | | | Letters, 116, 356-358. |
| Zr—O | 597 | 597 | Hajipour, A. R. (2014). Synthesis and characterization of |
| vibration | | | hexagonal zirconium phosphate nanoparticles. Materials |
| | | | Letters, 116, 356-358. |
| P—O | 521 | 525 | Kalita, H. (2016). Sonochemically synthesized |
| deformation | | | biocompatible zirconium phosphate nanoparticles for pH- |
| | | | sensitive drug delivery application. Materials Science and |
| | | | Engineering: C, 60, 84-91. |
| Zr—O | 415 | 444 | Kalita, H. (2016). Sonochemically synthesized |
| vibration | | | biocompatible zirconium phosphate nanoparticles for pH- |
| | | | sensitive drug delivery application. Materials Science and |
| | | | Engineering: C, 60, 84-91. |

Carbon-associated bands were not observed in the IR spectra and the absence of carbon was confirmed by elementary analysis (CHN).

The stability of the product in Fetal Bovine Serum (FBS) was tested. The product was mixed with FBS 100%, at a [Hf]=5.5 mM. The solution was incubated at 37° C. for 15 days and samples were analyzed by XRD at day 15. The layered structure was maintained after incubation in FBS.

### Example 2: Zr Oxide/Zr Phosphate Porous Structure

### Synthesis:

The Zr oxide/Zr phosphate product was prepared with the intention of intercalating zirconium phosphate layers with zirconium oxide. The protocol was adapted from Xiao, J (1999). Preparation and properties of zirconia-pillared zirconium phosphate and phenylphosphonate. Applied Catalysis A: General, 181,313-322

A suspension of Example 1 in ethylamine was obtained by adding dropwise a 0.1 mol/l ethylamine solution up to 100 ml/g ZrP (step 1, exfoliation). After aging for 24 h at ambient temperature, 400 ml of a freshly prepared ZrOCl₂.8H₂O (0.025 M, from Sigma-Aldrich®) precursor solution was added slowly to the suspension. The resulting new suspension was refluxed at 60° C. for 24 h, centrifuged, and washed with water (step 2, precursor salt condensation). The wet precipitate was dried at 60° C. overnight.

### Characterization:

The zirconium and phosphorus content of the powder was determined using Inductively Coupled Plasma-Optical Emission Spectrometry (ICP-OES). The ratio Zr/P was 1.3, confirming the introduction of further zirconium into the zirconium phosphate structure of starting material (Example 1).

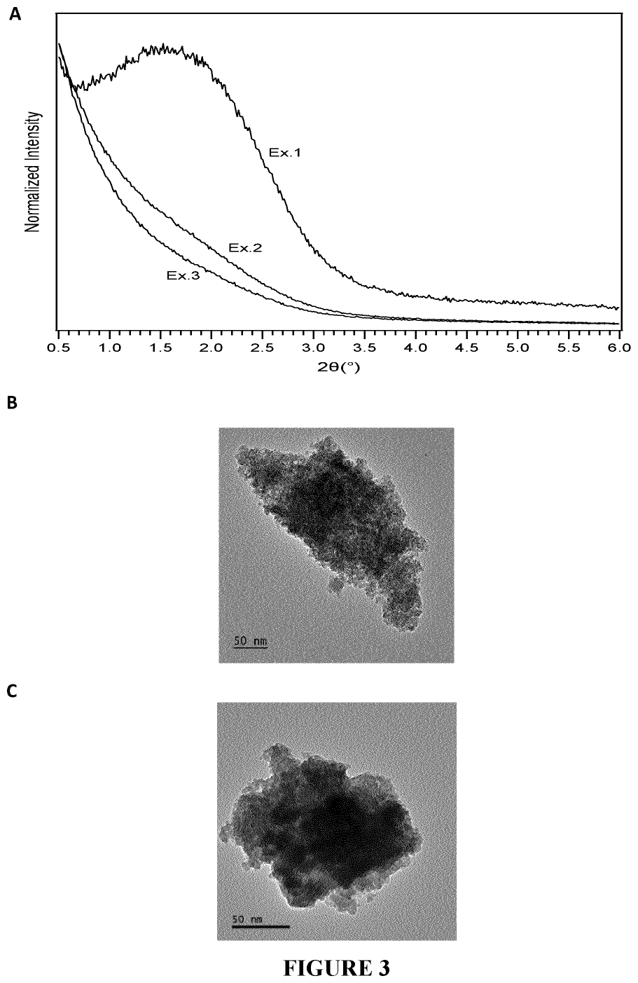

The X-Ray Diffraction (XRD) pattern of the powder showed the deformation of the diffraction peak at low angle observed in the XRD pattern of Example 1, which correlates with exfoliation in step 1 (FIG. 3A).

The Transmission Electron Microscopy (TEM) micrograph at high resolution is shown in FIG. 3.B.

Less than 0.5% carbon was detected by elemental analysis (CHN).

### Example 3: Hf Oxide/Zr Phosphate Porous Structure

### Synthesis:

The Hf-oxide/zirconium phosphate product was prepared with the intention of intercalating zirconium phosphate layers with hafnium oxide. The protocol was adapted from Xiao, J (1999). Preparation and properties of zirconia-pillared zirconium phosphate and phenylphosphonate. Applied Catalysis A: General, 181,313-322.

A suspension of Example 1 in ethylamine was obtained by adding dropwise a 0.1 mol/1 ethylamine solution up to 100 ml/g ZrP (step 1, exfoliation). After aging for 24 h at ambient temperature, 400 ml of a freshly prepared HfOCl₂·8H₂O (0.025 M, from Sigma-Aldrich®) precursor solution was added slowly to the suspension. The new suspension was refluxed at 60° C. for 24 h, centrifuged, and washed with water (step 2, precursor salt condensation). The wet precipitate was dried at 60° C. overnight.

### Characterization:

The zirconium, phosphorus and hafnium content of the powder were determined using Inductively Coupled Plasma-Optical Emission Spectrometry (ICP-OES). The ratio Zr/P was 0.8 and the ratio Zr/Hf was 1.5, these ratios confirmed the introduction of hafnium into zirconium phosphate structure of starting material (Example 1).

The X-Ray Diffraction (XRD) pattern of the powder showed the deformation of the diffraction peak at low angle observed in the XRD pattern of Example 1, which correlates with exfoliation in step 1 (FIG. 3A).

The BET specific surface area was estimated as for Example 1. The isotherm corresponds to a mesoporous material. The specific surface area of the powder was measured to be 171 m²/g and the pore diameter is 2.5 nm.

The Transmission Electron Microscopy (TEM) micrograph (FIG. 3C) at high resolution confirmed the porous structure found by BET. The presence of hafnium was confirmed by Energy Dispersive X-Ray (EDX) analysis.

The InfraRed spectra (IR) results are detailed in Table 3 below. The presence of Zr—O stretching deformation, P—O stretching vibration and P—O—H stretching vibration confirmed the metal phosphate composition.

TABLE 3

| | | Reference | |
|---|---|---|---|
| | Wavenumber | Wavenumber | |
| IR Band | (cm⁻¹) | (cm⁻¹) | References |
| P—O | 1051 | 1042 (Zr- | Um, W. (2007). Synthesis of nanoporous zirconium |
| vibration | | phosphate) | oxophosphate and application for removal of U |
| | | | (VI). Water Research, 41(15), 3217-3226. |
| | | 1060 (Hf- | Dushin, R. B. (1977). IR spectroscopic study of the |
| | | phosphate) | mechanism of ion exchange on amorphous hafnium |
| | | | phosphate. Bulletin of the Academy of Sciences of the |
| | | | USSR, Division of chemical science, 26(3), 469-472. |
| Zr—O | 593 | 597 | Hajipour, A. R., (2014). Synthesis and characterization |
| vibration | | | of hexagonal zirconium phosphate |
| | | | nanoparticles. Materials Letters, 116, 356-358. |
| P—O | 520 | 521 (in Zr | Kalita, H. (2016). Sonochemically synthesized |
| deformation | | phosphate) | biocompatible zirconium phosphate nanoparticles for |
| | | | pH-sensitive drug delivery application. Materials |
| | | | Science and Engineering: C, 60, 84-91. |
| | | 522 (in Hf | Dushin, R. B. (1977). IR spectroscopic study of the |
| | | phosphate) | mechanism of ion exchange on amorphous hafnium |
| | | | phosphate. Bulletin of the Academy of Sciences of the |
| | | | USSR, Division of chemical science, 26(3), 469-472. |
| Zr—O and | 429 | 444 (Zr—O) | Kalita, H. (2016). Sonochemically synthesized |
| Hf—O | | | biocompatible zirconium phosphate nanoparticles for |
| vibrations | | | pH-sensitive drug delivery application. Materials |
| | | | Science and Engineering: C, 60, 84-91. |
| | | 417 (Hf—O) | Dushin, R. B. (1977). IR spectroscopic study of the |
| | | | mechanism of ion exchange on amorphous hafnium |
| | | | phosphate. Bulletin of the Academy of Sciences of the |
| | | | USSR, Division of chemical science, 26(3), 469-472. |

Less than 0.5% carbon was detected by elemental analysis (CHN).

### Example 4: Porous Hafnium Oxide

### Synthesis:

Porous hafnium oxide was obtained by hydrolysis/condensation of HfCl₄. The protocol was adapted from Yang, P (1998). Generalized syntheses of large-pore mesoporous metal oxides with semicrystalline frameworks. Nature, 396(6707), 152-155.

0.6 g of Poly(ethylene glycol)-block-poly(propylene glycol)-block-poly(ethylene glycol) (pluronic P-123-Sigma) was dissolved in 6 mL of ethanol. To this solution, 0.01 mol of HfCl₄ (Framatome, Courbevoie, France) dissolved in butanol was added with vigorous stirring for 60 min. The resulting solution was left to dry in an open Petri dish at 40° C. for 7 days. The resultant solid was then washed with ethanol to remove surfactant, and dried overnight at 65° C.

### Characterization:

The hafnium content of the powder (0.55 gHf/g of powder) was determined using Inductively Coupled Plasma-Optical Emission Spectrometry (ICP-OES).

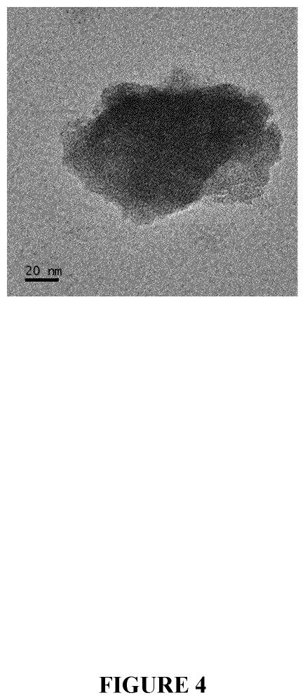

The Transmission Electron Microscopy (TEM) micrograph (FIG. 4) at high resolution confirmed the porous structure.

Less than 1.5% Carbon was detected by elemental analysis (CHN).

### Example 5: Mesoporous Hafnium Oxide

### Synthesis:

Mesoporous hafnium oxide was obtained by hydrolysis/condensation of HfCl₄. The protocol was adapted from Yang, P (1998). Generalized syntheses of large-pore mesoporous metal oxides with semicrystalline frameworks. Nature, 396(6707), 152-155.

0.6 g of Poly(ethylene glycol)-block-poly(propylene glycol)-block-poly(ethylene glycol) (pluronic P-123—Sigma-Aldrich®) was dissolved in 6 mL of ethanol. To this solution, 0.01 mol of HfCl₄ (Framatome) dissolved in butanol was added with vigorous stirring for 60 min. The resulting solution was left in an open Petri dish at 40° C. for 7 days. The powder was then calcined at 400° C. for 5 h to remove the surfactant.

### Characterization:

The hafnium content of the powder (0.71 g Hf/g of powder) was determined using Inductively Coupled Plasma-Optical Emission Spectrometry (ICP-OES).

The X-Ray Diffraction (XRD) pattern of the calcined powder showed a single broad diffraction peak in the low angle region. The pattern showed a broad diffraction peak at low angle (2 θ=0.5°) with a corresponding d₁₀₀ value of 17.6 nm.

The BET specific surface area was estimated. The isotherm corresponds to a mesoporous material. The specific surface area of the powder was measured to be 115 m²/g and the pore diameter is 5.0 nm.

The Transmission Electron Microscopy (TEM) micrograph (FIG. 5A) at high resolution also confirmed the porous structure.

The absence of carbon was confirmed by elementary analysis (CHN).

The stability of the product in Fetal Bovine Serum (FBS) was tested. The product was mixed with FBS 100%, at a [Hf]=7 mM. The solution was incubated at 37° C. for 7.5 and 15 days. The samples were analyzed by XRD after incubation. The layered structure was maintained after incubation in FBS for 15 days (FIG. 5B).

### Example 6: Porous Hafnium Phosphate

### Synthesis:

Porous hafnium phosphate was obtained by precipitation using a hafnium carbonate complex and diammonium hydrogen phosphate in a basic medium. The protocol was adapted from Tarafdar, A., (2006). Synthesis of spherical mesostructured zirconium phosphate with acidic properties. Microporous and mesoporous materials, 95(1-3), 360-365.

0.2 g of HfOCl₂·xH₂O (Sigma-Aldrich®) was dissolved in 100 mL of distilled water. 0.5 g of (NH₄)₂CO₃ (Sigma-Aldrich®) was added under stirring until a clear solution was obtained. 0.2 g of phosphate (NH₄)₂HPO₄ (Supelco®) was added and dissolved into the solution.

0.08 g of (1-Tetradecyl)trimethylammonium bromide (TTBr, from Alfa Aesar, Haverhill, Massachusetts, USA) was added to the solution with continuous stirring. The resultant clear solution was kept in an oven at 80° C. for 3 days in a closed polypropylene tube for complete precipitation and was then aged in a Teflon autoclave at 90° C. for 2 days, and then at 120° C. for 24 h The sample was cooled and then washed three times with distilled water. The solid was dried at 65° C. overnight and calcined at 540° C. for 6 h.

### Characterization:

The hafnium and phosphorus content of the powder was determined using Inductively Coupled Plasma-Optical Emission Spectrometry (ICP-OES). The ratio Hf/P was 0.6.

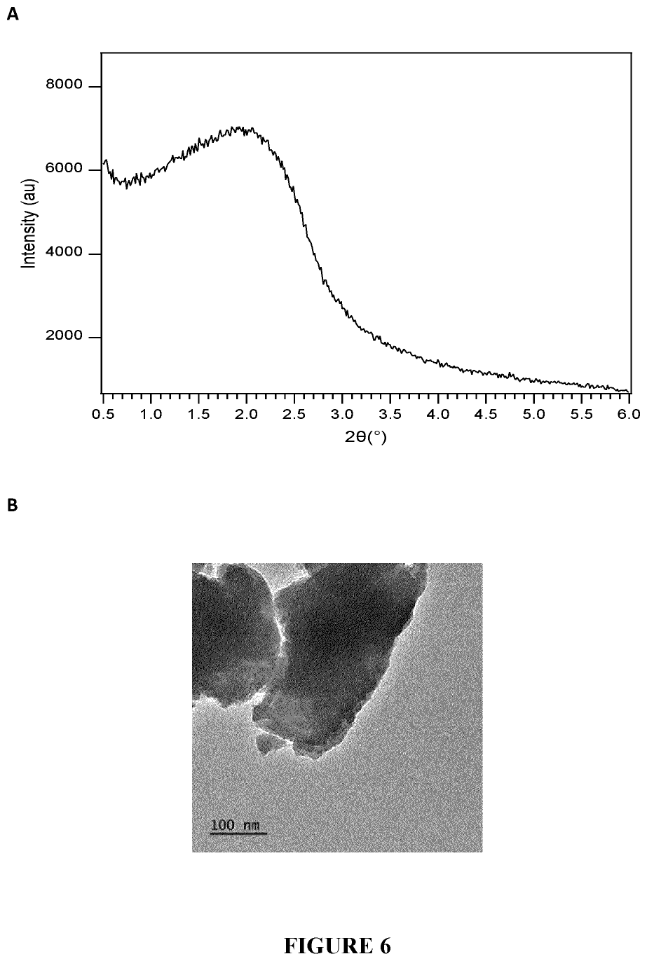

The X-Ray Diffraction (XRD) pattern of the calcined powder showed a single reflection at low angle region (FIG. 6A). The pattern showed a broad diffraction peak at low angle (2 θ=2.0°) with a corresponding d₁₀₀ value of 4.4 nm.

The Transmission Electron Microscopy (TEM) micrograph (FIG. 6.B) at high resolution confirmed the porous structure found by XRD.

The InfraRed spectra (IR) results are detailed in Table 4 below. The presence of Hf—O stretching deformation, P—O stretching vibration and P—O—H stretching vibration confirmed the metal phosphate composition.

TABLE 4

| | | Reference | |
|---|---|---|---|
| | Wavenumber | Wavenumber | |
| IR Band | (cm⁻¹) | (cm⁻¹) | References |
| P—O—H | 2412 | 2400 | Dushin, R. B. (1977). |
| vibration | | | IR spectroscopic study |
| P—O—H | 1241 | 1240 | of the mechanism of |
| vibration | | | ion exchange on amorphous |
| P—O | 1056 | 1060 | hafnium phosphate. |
| vibration | | | Bulletin of the Academy |
| Hf—O | 604 | 617 | of Sciences of the USSR, |
| vibration | | | Division of chemical |
| P—O | 521 | 522 | science, 26(3), 469-472. |
| deformation | | | |
| Hf—O | 414 | 417 | |
| vibration | | | |

Carbon-associated bands were not observed in the IR spectra. The absence of carbon was confirmed by elementary analysis (CHN).

### Example 7: Porous Hafnium Phosphate

### Synthesis:

Porous hafnium phosphate was obtained by precipitation of Hf(SO₄)₂ followed by sulfate ion replacement with phosphate ions. The protocol was adapted from Um, W (2007). Synthesis of nanoporous zirconium oxophosphate and application for removal of U (VI). Water Research, 41(15), 3217-3226.

Two solutions were prepared by dissolving 1.8008 g Hf(SO₄)₂ (Alfa Aesar) (solution A) and 1.0262 g of octadecyltrimethylammonium bromide surfactant (OCTBr from Sigma-Aldrich®) (solution B) in 20 g of distilled water. Solution A was then added dropwise to solution B under gentle stirring (1200 rpm). The resulting mixture was stirred for 2 h at room temperature and then heated to 90° C. for 2 days in a polypropylene bottle. The precipitate was then filtered and mixed with 40 ml of an aqueous solution of phosphoric acid (H₃PO₄, 0.5 M, from Supelco®) under vigorous stirring for 24 h at room temperature. The final product was filtered using a 0.45-mm membrane, washed with distilled water, and dried at room temperature for 48 h. The surfactant was removed by calcination at 500° C. in a furnace for 7 h.

### Characterization:

The hafnium and phosphorus content of the powder was determined using Inductively Coupled Plasma-Optical Emission Spectrometry (ICP-OES). The ratio Hf/P was 0.5.

The X-Ray Diffraction (XRD) pattern of the calcined powder showed a single reflection at low angle region. The pattern showed a broad diffraction peak at low angle (2 θ=2.3°) with a corresponding d₁₀₀ value of 3.9 nm.

The BET specific surface area was estimated. The isotherm corresponds to a porous material. The specific surface area of the powder was measured to be 340 m²/g and the pore diameter is 2.0 nm, lying between microporosity and mesoporosity.

The Transmission Electron Microscopy (TEM) micrograph (FIG. 7.A) at high resolution also confirmed a porous structure.

The InfraRed spectra (IR) results are detailed in Table 5 below. The presence of Hf—O stretching deformation, P—O stretching vibration and P—O—H stretching vibration confirmed the metal phosphate composition.

TABLE 5

| | | Reference | |
|---|---|---|---|
| | Wavenumber | Wavenumber | |
| IR Band | (cm⁻¹) | (cm⁻¹) | References |
| P—O—H | 2420 | 2400 | Dushin, R. B. (1977). |
| vibration | | | IR spectroscopic study |
| P—OH | 1267 | 1240 | of the mechanism of |
| P—O | 1100 | 1060 | ion exchange on amorphous |
| vibration | | | hafnium phosphate. |
| Hf—O | 601 | 617 | Bulletin of the Academy |
| vibration | | | of Sciences of the USSR, |
| P—O | 519 | 522 | Division of chemical |
| deformation | | | science, 26(3), 469-472. |
| Hf—O | 425 | 417 | |
| vibration | | | |

Carbon-associated bands were not observed in the IR spectra and less than 0.5% carbon was detected by elemental analysis (CHN).

The stability of the product in Fcetal Bovine Serum (FBS) was tested. The product was mixed with FBS 100%, at a [Hf]=6.5 mM. The solution was incubated at 37° C. for 7.5 and 15 days. The samples were analyzed by XRD after incubation. The layered structure was maintained after incubation in FBS at 15 days (FIG. 7B).

The stability of the product under irradiation was tested. The product was mixed with water, at a [Hf]=7.5 mM. The solution was irradiated at 2 Gy and 20 Gy in a T25 flask and samples were analyzed by XRD. The layered structure was maintained after irradiation (FIG. 7C).

### Example 8: Mesoporous Zirconium Phosphate

### Synthesis:

Mesoporous zirconium phosphate was obtained using the sol-gel method. The protocol was adapted from Jiménez-Jiménez, J., (1998). Surfactant-Assisted Synthesis of a Mesoporous Form of Zirconium Phosphate with Acidic Properties. Advanced Materials, 10(10), 812-815.

An aqueous solution of Hexadecyltrimethylammonium bromide (CTAB from Sigma) (250 g/L) was mixed with orthophosphoric acid (85 wt.-%, Supelco) (P/CTAB molar ratio=1). The CTAB solution was then aged for at least 30 min before adding zirconium n-propoxide (Zr(OC₃H₇)₄, 70 wt.-% solution in 1-propanol, (Sigma), in a molar ratio P/Zr=2. The gel that immediately formed was stirred at room temperature for 3 days. The solid was then recovered by filtration, washed with ethanol, and dried at 60° C. overnight. The surfactant was removed by calcination at 540° C. for 6 h.

### Characterization:

The zirconium and phosphorus content of the powder was determined using Inductively Coupled Plasma-Optical Emission Spectrometry (ICP-OES). The ratio Zr/P was 0.5.

The BET specific surface area was estimated as for Example 1. The isotherm corresponded to a mesoporous material. The specific surface area of the powder was measured to be 132 m²/g and the pore diameter is 5.0 nm.

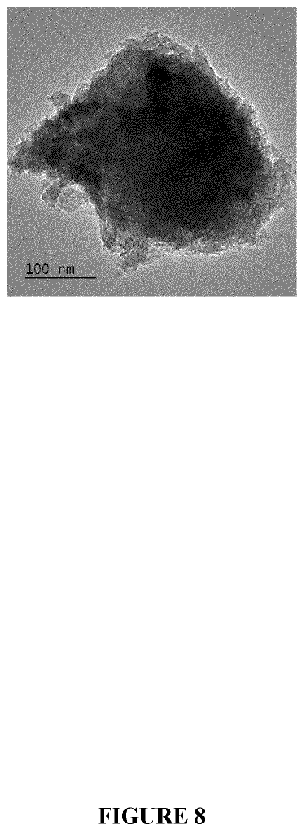

The Transmission Electron Microscopy (TEM) micrograph (FIG. 8) at high resolution confirmed the porous structure found by BET.

The InfraRed spectra (IR) results are detailed in Table 6 below. The presence of Zr—O stretching deformation, P—O stretching vibration and P—O—H stretching vibration confirmed the metal phosphate composition.

TABLE 6

| | | Reference | |
|---|---|---|---|
| | Wavenumber | Wavenumber of | |
| IR Band | (cm⁻¹) | the (cm⁻¹) | References |
| P—O—H | 2389 | 2350 | Um, W. (2007). Synthesis of nanoporous |
| vibration | | | zirconium oxophosphate and application for |
| | | | removal of U (VI). Water Research, 41(15), |
| | | | 3217-3226. |
| P—O—H | 1255 | 1250 | Xiao, J. (1999). Preparation and properties of |
| vibration | | | zirconia-pillared zirconium phosphate and |
| | | | phenylphosphonate. Applied Catalysis A: |
| | | | General, 181(2), 313-322. |
| P—O | 1099 | 1000-1200 | Huang, H. (2020). Exfoliation and |
| vibration | | | functionalization of α-zirconium phosphate in |
| | | | one pot for waterborne epoxy coatings with |
| | | | enhanced anticorrosion performance. Progress |
| | | | in Organic Coatings, 138, 105390. |
| Zr—O | 589 | 597 | Hajipour, A. R. (2014). Synthesis and |
| vibration | | | characterization of hexagonal zirconium |
| | | | phosphate nanoparticles. Materials Letters, 116, |
| | | | 356-358. |
| P—O | 521 | 525 | Kalita, H. (2016). Sonochemically synthesized |
| deformation | | | biocompatible zirconium phosphate |
| | | | nanoparticles for pH-sensitive drug delivery |
| | | | application. Materials Science and |
| | | | Engineering: C, 60, 84-91. |
| Zr—O | 415 | 444 | Kalita, H. (2016). Sonochemically synthesized |
| vibration | | | biocompatible zirconium phosphate |
| | | | nanoparticles for pH-sensitive drug delivery |
| | | | application. Materials Science and |
| | | | Engineering: C, 60, 84-91. |

Carbon-associated bands were not observed in the IR spectra and less than 0.5% carbon was detected by elemental analysis (CHN).

### Reference Example 9: Porphyrin Based Hafnium Organic Framework (Hf—PO)

### Synthesis:

Hf—PO was synthesized with a solvothermal reaction between HfCl₄ (50 μM; from Framatome) and 4,4′,4″,4″′-(Porphine-5,10,15,20-tetrayl)tetrakis(benzoic acid) (PO; 20 μM; from Sigma) in N,N-Diethylformamide (DEF; Sigma) at 120° C. for 48 h, with formic acid as modulator. The powder was washed by centrifugation with 1% triethylamine in ethanol (v/v), and ethanol, and dried at 65° C.

### Characterization:

The hafnium content of the powder (0.33 g Hf/g of powder) was determined using Inductively Coupled Plasma-Optical Emission Spectrometry (ICP-OES).

The X-Ray Diffraction (XRD) pattern of the powder showed the same diffraction peaks as those published reference Metal Organic Framework structure, MOF-545 [Morris, W. (2012) Synthesis, Structure, and Metalation of Two New Highly Porous Zirconium Metal-Organic Frameworks. Inorg. Chem, 51, 6443-6445.].

The Transmission Electron Microscopy (TEM) micrograph (FIG. 9.A) at high resolution confirmed the porous structure.

The stability of the product in Fetal Bovine Serum (FBS) was tested. The product was mixed with FBS 100%, at a [Hf]=10 mM. The solution was incubated at 37° C. for 10 days. The sample was analyzed by XRD after incubation. A broadening of the diffraction peaks was observed after incubation in FBS (FIG. 9B), which indicates a loss of product crystallinity (3-D structure).

### Example 10: In Vitro Efficacy of Products Described in Examples 1 and 4

### In Vitro Experiments:

The cell viability assay is based on the cleavage of the tetrazolium salt, WST-1 (4-[3-(4-iodophenyl)-2-(4-nitrophenyl)-2H-5-tetrazolio]-1,3-benzene disulfonate) (Sigma-Aldrich®), by mitochondrial dehydrogenases in viable cells to form highly water-soluble formazan. Vehicle (Ethanol or Ethanol/water (1/1)), was used for radiotherapy alone control. Assays were performed on mouse CT26 cell line in 96-well microplates. Two thousand (2000) cells (10 000 cells/ml, 200 μL/well) were incubated in the presence of increasing concentrations of product. A single irradiation dose of 2 Gy was applied after 24 h of cell incubation with the product. Irradiation was performed with a 320 kV irradiator (Model X-Rays XRAD 320). The WST-1 assay is measured after a 96 h post-irradiation period (a control without irradiation was also performed). Cells were incubated with 10 μL of WST-1 to achieve 1:10 final dilution. After 45 min, supernatants were collected and the absorbance of the converted dye was measured at a wavelength of 450 nm. Cell viability was expressed as % of the control (non-irradiated, untreated cells).

A marked radio enhancement effect on the CT26 cells was observed, with increasing concentrations of both Example 1 and Example 4, when activated by X-rays (FIGS. 10 and 11). Full boxes represent cell viability without irradiation, while hatched boxes represent cell viability after an irradiation dose of 2 Gy. The dashed line corresponds to the effect of radiotherapy alone on cell viability.

FIG. 10B shows that, at 325 μM of product Example 1, 22% cell viability is obtained after irradiation at 2 Gy. FIG. 10A shows that, at 650 μM concentration, 17% cell viability is obtained after irradiation. A 37% viability was obtained for the irradiated control sample (vehicle). A greater amount of cell killing occurs in the presence of Example 1.

FIG. 11B shows that, at 705 μM of product Example 4, 56% cell viability is obtained after irradiation at 2 Gy. FIG. 11A indicates that, at 1410 μM concentration, 53% cell viability is obtained after irradiation. A 66% viability was obtained for the irradiated control sample (vehicle). Thus, a greater amount of cell killing occurs in the presence of Example 4.

### Example 11: In Vitro Clonogenic Assay of Product Described in Example 9 (MOF)

The clonogenic cell survival assay evaluates cell survival by the ability of a single cell to form colony after a treatment that might influence the proliferation rate and/or can cause senescence, reproductive cell death or apoptosis.

CT26 cells were seeded in triplicate into 6 well plates in a range of 100-1000 cells/well depending on the test condition and radiation dose. Once cells were attached to the plate, they were exposed to the product of Example 9 (100 μM). Two different doses (2 and 4 Gy) of X-rays were delivered to the cells 15-16 hours post-treatment. After irradiation, the Example 9 product (nanoparticles) were removed, and fresh medium added. The cells were cultured up to 6±1 days at 37° C. under 5% CO₂. The colonies were fixed and stained with crystal violet (Sigma-Aldrich® HT90132) (25% in EtOH) 1-2 ml/well. All colonies of 50 cells or more were then counted. At a concentration 100 μM the MOF product of Example 9 showed no radio enhancement effect at either 2 Gy or 4 Gy.

1-15. (canceled)

16. A method of altering or destroying target cancerous cells in a mammal comprising administering porous, high-Z and carbon-free particles or a composition comprising said particles and a pharmaceutically acceptable carrier, vehicle or support to a mammal in need of treatment, and exposing the cells to ionizing radiation, wherein the porous, high-Z and carbon-free particles have internal pores of longest dimension of between 0.5-50 nm, and wherein the particles comprise:
a) a high-Z metal phosphate, a high-Z metal oxo phosphate, a high-Z metal oxide or a high-Z mixed metal oxide, wherein the high-Z metal phosphate, high-Z metal oxo phosphate, high Z metal oxide or high-Z mixed metal oxide comprises at least one high-Z metal element, wherein the Z value of the at least one high-Z metal element is of at least 40; or
b) a plurality of high-Z metal phosphate layers, wherein the Z value of the high-Z metal element of each metal phosphate layer is of at least 40 and wherein each high-Z metal phosphate layer is connected to an adjacent high-Z metal phosphate layer via a carbon free linker;
and optionally, a biocompatible surface coating.

17. The method according to claim 16, wherein the BET surface area is at least 50 m²/g.

18. The method according to claim 16, wherein the particles have a pore of internal width between 2 and 30 nm.

19. The method according to claim 16, wherein the high-Z metal is selected from a lanthanide, tantalum (Ta), tin (Sn), zirconium (Zr), cerium (Ce) hafnium (Hf), tungsten (W), niobium (Nb), titanium (Ti) or rhenium (Re).

20. The method according to claim 16, wherein the particles comprise a high-Z metal oxide or a mixed metal oxide of respective composition MₓO_{y}, and MₓM′_{z}O_{y}, wherein M and M′ are metal elements chosen independently from a lanthanide element, zirconium (Zr) and hafnium (Hf).

21. The method according to claim 16, wherein the metal phosphate is selected from a hafnium phosphate, a zirconium phosphate and a lanthanide phosphate, or the metal oxo phosphate is a hafnium oxo phosphate or a zirconium oxo phosphate, or the metal oxide or the mixed metal oxide is selected from Nb₂O₅, Ta₂O₅, WO₃, HfO₂, SnO₂, ZrTiO₄, and ZrW₂O₈, or the metal phosphate layers are hafnium phosphate layers, zirconium phosphate layers or a mixture thereof.

22. The method according to claim 16, wherein at least part of the porous structure of the particles is occupied by at least one therapeutic agent, selected from an immunotherapeutic agent, a cytotoxic agent, a targeted therapeutic agent, a photothermal agent, a photodynamic agent and any mixture thereof.

23. The method according to claim 16, wherein the particles further comprise a targeting agent that recognizes an element present on a cancer cell and comprise a peptide, an oligopeptide, a protein, a nucleic acid, a hormone, a vitamin, an enzyme, the ligand of a tumor antigen, hormone receptor, cytokine receptor or growth factor receptor.

24. The method according to claim 16, wherein the cells are exposed to ionizing radiation from a radiotherapy regimen selected from the group consisting of a conventional fractionation regimen, an hyperfractionation regimen, an (accelerated) hypofractionation regimen and a stereotactic ablative body radiotherapy (SBAR) regimen.

25. The method according to claim 24, wherein the fractionated regimen is given over more than ten days.

26. The method according to claim 16, wherein the target cancerous cells belong to a solid malignant tumor selected from a skin cancer, a central nervous system cancer, a head and neck cancer, a lung cancer, a liver cancer, a breast cancer, a gastrointestinal cancer, a male genitourinary cancer, a gynecologic cancer, an adrenal and/or retroperitoneal cancer, a sarcoma and a pediatric cancer.

27. A composition comprising porous, high-Z and carbon-free particles and a pharmaceutically acceptable carrier, vehicle or support, wherein the porous, high-Z and carbon-free particles have internal pores of longest dimension of between 0.5-50 nm, and wherein the particles comprise:
a) a high-Z metal phosphate, a high-Z metal oxo phosphate, a high-Z metal oxide or a high-Z mixed metal oxide, wherein the high-Z metal phosphate, high-Z metal oxo phosphate, high Z metal oxide or high-Z mixed metal oxide comprises at least one high-Z metal element, wherein the Z value of the at least one high-Z metal element is of at least 40; or
b) a plurality of high-Z metal phosphate layers, wherein the Z value of the high-Z metal element of each metal phosphate layer is of at least 40 and wherein each high-Z metal phosphate layer is connected to an adjacent high-Z metal phosphate layer via a carbon free linker;
and optionally, a biocompatible surface coating.

28. The composition according to claim 27, said composition comprising particles selected from particles a), b) and a mixture of both a) and b).

29. The composition according to claim 27, wherein the vehicle or support is selected from a liposome, viral vector, viral-like particle, albumin containing carrier, inorganic polymer and organic polymer.
